(19) United States
(12) Patent Application Publication
(10) Pub. No.: US 2016/0136078 A1
(43) Pub. Date: May 19, 2016

(54) Method for Stabilizing Ascorbic Acid Derivatives and the Application Thereof

(71) Applicant: CORUM INC., Taipei City (TW)

(72) Inventors: Lin-Chao Chen, Taipei City (TW); Yu-Ling Shiu, Taipei City (TW); Chia-Pei Lee, Taipei City (TW); Chao-Yang Lee, Taipei City (TW)

(21) Appl. No.: 15/008,120

(22) Filed: Jan. 27, 2016

Related U.S. Application Data
(63) Continuation-in-part of application No. 13/689,971, filed on Nov. 30, 2012, now abandoned.

(51) Int. Cl. A61K 8/49; A61K 8/365; A61Q 19/00; A61K 8/39; A61K 8/34; A61K 8/06; A61K 8/55

(52) U.S. Cl. CPC A61K 8/4973; A61K 8/062; A61K 8/365; A61K 8/55; A61K 8/39; A61K 8/345; A61Q 19/00; A61K 2800/52; A61K 2800/10; A61K 2800/74; A61K 2800/592

(57) ABSTRACT

This invention discloses a method for stabilizing ascorbic acid derivatives and the application thereof. The mentioned method comprises mixing ascorbic acid derivative with a non-water-in-oil composition, and the composition comprises buffer, phosphonic acid derivative and at least one alcohol. The yellowish and degradation of ascorbic acid derivative can be efficiently decreased by the mentioned method. Moreover, the mentioned method can be used in topical composition, such as toner, serum, lotion, cream.

### CROSS REFERENCE TO RELATED APPLICATIONS

This application is a Continuation In Part of applicant's earlier application Ser. No. 13/689,971, filed Nov. 30, 2012.

### BACKGROUND OF THE INVENTION

1. Field of the Invention

The present invention is generally related to an ascorbic acid derivatives composition, and more particularly to a method for stabilizing ascorbic acid derivatives and the application thereof.

2. Description of the Prior Art

Ascorbic acid, a.k.a. (also called as) vitamin C, is a water-soluble antioxidant. In addition to its anti-oxidation property, vitamin C is not only recognized for its ability to protect human body from harmful effects of free radicals and environmental pollutants (including CO, hydrocarbons, pesticides and heavy metals), but also for its property to protect DNA of human cells from the damage caused by free radicals and mutagens. Another important function of vitamin C is to strengthen skin tissues through the formation and maintenance of collagens, which helps reduce the expression of wrinkles and delay skin ageing. Especially in the cosmetic industry, vitamin C is also identified to help in the metabolism of tyrosine by inhibiting melanization and preventing skin darkening, thus making it an effective whitening/lightening agent for human skin. Furthermore, vitamin C plays a significant role in many biological functions of human body, as reported in the article “Biological Significance of Ascorbic Acid (Vitamin C) in Human Health—A Review”, published in Pakistan Journal of Nutrition 3(1):5-13, 2004.

Despite all its benefits, vitamin C is extremely unstable; it can be easily oxidized and degraded by oxygen, light, alkali, metals, and high temperature.

In order to stabilize ascorbic acid, a special container for a composition containing ascorbic acid and a hydrophilic carrier which are packaged separately but mixed together upon use was developed in U.S. Pat. No. 6,010,706. This technology of mixing two components, from 0.001 to 0.1 grams of ascorbic acid per gram of carrier, ensures that ascorbic acid does not break and remains stable at room temperature for at least one week. In other words, if this container is not being used, the vitamin C stability will be very poor.

U.S. Pat. No. 5,140,043 discloses a composition of ascorbic acid in water and propylene glycol with a pH value of less than 3.5. However, such a low pH could severely irritate human skin, and in some countries, cosmetic laws even prohibit the use of pH lower than 3.5.

U.S. Pat. No. 5,736,567 discloses a composition which contains ascorbic acid dissolved in water and at least one alcohol, forming an aqueous phase, wherein alcohol is present in a quantity that is effective for obtaining a water activity value of ≦0.85. The amount of alcohol(s) used is preferably 45-80% by weight, which is considered a large amount of alcohol(s) in the composition.

U.S. Pat. No. 8,053,469 indicates a production technology that helps stabilize high content of ascorbic acid. This process involves sequential additions of vitamin C, ethoxydiglycol and propylene glycol into the initial solution of vitamin C dissolved in approx. 10% water; and vitamin C at high content has to be divided and added into the solution in several sequences. Nevertheless, the solution also contains a large amount of propylene glycol.

U.S. Pat. No. 6,087,393 discloses a stabilized system of ascorbic acid in a mixed glycol solution. This mixed glycol carrier contains a mixture of propylene glycol and butylene glycol at 25-80% by weight and 5-30% by weight, respectively. Likewise, this composition also contains a high level of propylene glycol.

The four U.S. patents mentioned above can help to improve the stability of vitamin C in various formulations, but there are still some concerns over the use of high concentration of propylene glycol in cosmetic formulations. The North American Contact Dermatitis Group currently recommends a 10% aqueous propylene glycol solution for patch testing, because allergic sensitization has been confirmed by several repeated patch tests, usage tests and oral provocation tests in selected cases. In particular, a significant number of reactions to propylene glycol represent a primary irritant effect. From the studies listed in the article “Propylene glycol dermatitis”, published in Journal of the American Academy of Dermtaology 1991; 24:90-5, it is also clear that there are an increasing amount of irritant reactions when propylene glycol is used in higher concentrations. However, controversies still exist on the potential of allergic sensitizations and irritant reactions caused by this substance.

Furthermore, U.S. Pat. No. 6,110,476 describes a synergistic system based on a phosphonic acid derivative and metabisulfite to stabilize ascorbic acid. However, sodium metabisulfite has been reported as a contact allergen and also as a cause of allergic contact dermatitis in the article “Sodium metabisulfite as a contact allergen—an example of a rare chemical mechanism for protein modification”, published in 2012 John Wiley & Sons A/S•Contact Dermatitis, 66, 123-127. This compound also has a faint SO₂ odor that is unpleasant and pungent to human noses.

3-O-ethyl ascorbic acid is a vitamin C derivative consisting of a conventional vitamin C structure and an additional ethyl group, which makes it more stable than vitamin C. 3-O-ethyl ascorbic acid is tested and recognized for its outstanding ability to inhibit free radical activity, inhibit tyrosinase activity, inhibit melanin production, stimulate collagen synthesis, protected DNA and clinically whiten/lighten/brighten skin tone. Many of these properties have been reported in details by Jill Hsu in the article “New multi-functional and stable vitamin C for skin lightening”, published in NutraCos Cosmetics May/August 2012, p. 6-7.

In addition, another important property of 3-O-ethyl ascorbic acid has been identified in U.S. Pat. No. 2003/0134264A1, which discloses a method of preventing darkening of skin or inhibiting melanization of melanin monomer and a polymerization inhibitor of biological dihydroxyindole compound. The polymerization inhibitor 3-O-ethyl ascorbic acid inhibits the polymerization of a biological dihydroxyindole compound, caused by long wavelength of UVA, and thus reduces melanization significantly.

Although 3-O-ethyl ascorbic acid has a better stability than ascorbic acid, the complete stability of this ascorbic acid derivative hasn't yet been proven and remains unknown up till now.

In view of the above matters, developing a novel method having the advantage of stabilizing ascorbic acid derivatives and being able to be used in topical composition is still an important task for the industry.

### SUMMARY OF THE INVENTION

In light of the above background, in order to fulfill the requirements of the industry, the present invention provides a novel method and the application thereof having the advantage of stabilizing ascorbic acid derivatives with mild condition, so that the mentioned method can be employed in topical composition, such as toner, serum, lotion, cream.

One objective of the present invention is to provide a method for stabilizing ascorbic acid derivatives to reduce the degradation of the ascorbic acid derivatives therein.

Another objective of the present invention is to provide a method for stabilizing ascorbic acid derivatives to minimize the color change of the ascorbic acid derivatives compositions.

Still another objective of the present invention is to provide a method for stabilizing ascorbic acid derivatives. The mentioned method does not employ high concentration alcohols therein, so that the method of this specification can be potentially employed in cosmetics and dermatologic fields without allergic sensitizations and irritant reactions.

Accordingly, the present invention discloses a method for stabilizing ascorbic acid derivatives and the application thereof. The mentioned method for stabilizing ascorbic acid derivatives is mixing ascorbic acid derivatives with a composition, wherein the composition comprises buffer, phosphonic acid derivative, and at least one alcohol. The alcohol must be compatible with water, be polar with one or more hydroxyl groups, and be acceptable for cosmetic use. According to this invention, the mentioned method can efficiently minimize the color change of the ascorbic acid derivatives solution, and efficiently reduce the degradation of the ascorbic acid derivatives. We find out that ascorbic acid derivatives can be separately stabilized by adjusting the pH value of the composition, adding few amount of phosphonic acid derivative, or adding few amount of at least one alcohol. Preferably, the method for stabilizing ascorbic acid derivatives can be potentially applied in cosmetics and dermatologic fields without allergic sensitizations and irritant reactions to human skin.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1 shows a bar chart of using different alcohols and different amount of alcohols for stabilizing ascorbic acid derivatives solution of this invention.

### DESCRIPTION OF THE PREFERRED EMBODIMENTS

What probed into the invention is a method for stabilizing ascorbic acid derivatives and the application thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses a method for stabilizing ascorbic acid derivatives. The mentioned method comprises mixing ascorbic acid derivatives with a composition, wherein said composition comprises buffer, phosphonic acid derivative, and at least one alcohol. The general formula of the mentioned ascorbic acid derivatives is as the following.

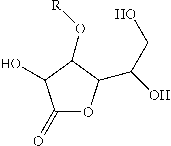

In the above-mentioned formula, R is selected from one of the group consisting of the following: C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group. In one preferred example of this embodiment, the mentioned ascorbic acid derivative is 3-O-ethyl ascorbic acid with the structure as following.

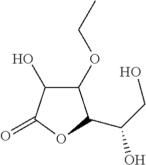

The mentioned buffer is employed to adjust pH of the composition. Preferably, pH of the composition is between 3.5 and 5.5. More preferably, pH of the composition is between 3.8 and 4.5. The mentioned buffer is selected from one of the group consisting of the following: citric acid/sodium citrate (pH 3.0-6.2), citric acid/sodium phosphate (pH 2.6-7.6), sodium acetate/acetic acid (pH 3.7-5.6). In one preferred example of this embodiment, the mentioned buffer is citric acid/sodium citrate (pH 3.0-6.2). The mentioned phosphonic acid derivative is selected from one of the group consisting of the following: N,N,N′,N′-ethylenediaminetetrakis(methylenephosphonic acid) hydrate (EDTMP), hexaMethylenediaminetetra(methylenephosphonic Acid) (HMDTMPA), Diethylene Triamine Penta(Methylene Phosphonic Acid) (DTPMPA) and the salts thereof.

The mentioned alcohol is selected from one or the combination of the group consisting of the following: ethanol, glycerin, propylene glycol, 1,3-propanediol, dipropylene glycol, butylene glycol, ethoxydiglycol, and polyethylene glycol (PEG). In one preferred example, the average molecular weight of polyethylene glycol is about from 100 to 600 g/mol.

In one preferred example of this embodiment, the mentioned method for stabilizing ascorbic acid derivatives comprises the ascorbic acid derivative from 0.01 to 10% of the total weight of the composition. Preferably, the quantity of the ascorbic acid derivative is from 0.1 to 4.0% of the total weight of the composition. In one preferred example of this embodiment, the mentioned method for stabilizing ascorbic acid derivatives comprises the phosphonic acid derivative from 0.01 to 1.0% of the total weight of the composition. Preferably, the quantity of the phosphonic acid derivative is from 0.1 to 0.5% of the total weight of the composition. In one preferred example of this embodiment, the mentioned composition for stabilizing ascorbic acid derivatives comprises the mentioned alcohol not more than 20% of the total weight of the composition. Preferably, the quantity of the alcohol is not more than 10% of the total weight of the composition. In the mentioned composition, the composition further comprises buffer and solvent so that the total weight of the composition approaches 100%. In one preferred example of this embodiment, the solvent is water.

In one preferred example of this embodiment, the mentioned composition is an oil-in-water composition (0/W). In another preferred example of this embodiment, the mentioned composition is a water-loving composition.

According to IUPAC definition, an emulsion is termed an oil/water (o/w) emulsion if the dispersed phase is an organic material and the continuous phase is water or an aqueous solution and is termed water/oil (w/o) if the dispersed phase is water or an aqueous solution and the continuous phase is an organic liquid (an “oil”).

The preferred examples of the structure and fabricating method for stabilizing ascorbic acid derivatives and the application thereof according to the invention are described in the following. However, the scope of the invention should be based on the claims, but is not restricted by the following examples.

In the following examples, the transmittance is measured by UV-Vis spectrophotometer. The measuring device is Thermo MULTISKAN GO, and the wavelength is set on 440 nm. The general measuring procedure is as the following. A cuvette loaded with distilled water is put into the device for calibration as zero. And then the cuvette loaded with sample is put into the device for measuring the absorbance at 25° C. The transmittance of the sample can be calculated by the following formula.

A=−log T

or written as:

T %=10^{-A+2}

Wherein A is absorbance, and T is transmittance (hereinafter presented transmittance as T %). When the measured transmittance of the sample is lower, the sample is more yellow.

The activity of ascorbic acid derivatives is also measured by HPLC (High Performance Liquid Chromatography) in this specification. The measuring device is Agilent 1260 HPLC: Quat pump/ALS/TCC/DAD; Column: Prodigy/ODS-3/00F-4097-E0/4.6*150 mm. A bi-solvent system is employed as the mobile phase, the flow rate is set as 1 mL/min, and the detector at 245 nm. In the bi-solvent system, solution A is 0.1% TFA (trifluoroacetic acid)/Acetonitrile, and solution B is 0.1% TFA/double distilled water. Each sample injection is 10 μL. The mobile phase is performed as gradient elution at 25° C., and the gradient program is as the following.

| Time (min) | Solution A (%) | Solution B (%) |
|---|---|---|
| 0.00 | 2 | 98 |
| 10.00 | 98 | 2 |
| 15.00 | 98 | 2 |
| 15.01 | 2 | 98 |
| 20.00 | 2 | 98 |

The total run time is 20 minutes for each injection. And the retention time of the sharp target peak appears on 5.4 minute, while the ascorbic acid derivative is 3-O-ethyl ascorbic acid. The integral of the target peak area is employed for representing the content of ascorbic acid derivative in the sample.

### Example 1

For testing the pH decline, 3-O-ethyl ascorbic acid is dissolved in water, and the aqueous solution is placed at 45° C. for 90 days. The test result is presented as the following Table 1. In Entry 1, 1 g 3-O-ethyl ascorbic acid was dissolved in purified water to 100 g form 2% (w/w) solution. In Entry 2, 2 g 3-O-ethyl ascorbic acid and 0.0007 g sodium citrate were dissolved in purified water to 100 g. In Entry 3, 2 g 3-O-ethyl ascorbic acid, 1.52 g sodium citrate and 0.926 g citric acid were dissolved in purified water to 100 g. In the above experiments, the total amount of the sample that contains the appropriate amount of the preservative.

As shown in Entry 3 in Table 1, buffer system is helpful to stabilize the pH of 3-O-ethyl ascorbic acid solution.

### Example 2

In this example, we try to find out the relationship between the pH value and the transmittance (color change) of ascorbic acid derivative solution. In this example, the following solutions were placed at 45° C. for 90 days, and the transmittance of the solutions on Day 0 and Day 90 were respectively detected. Table 2 presents the result of this example. In Entry 4, 0 g 3-O-ethyl ascorbic acid, 1.558 g sodium citrate and 0.993 g citric acid were dissolved in purified water to 100 g as the first blank experiment. The pH value of the mentioned first blank experiment is 4.49. In Entry 5, 0 g 3-O-ethyl ascorbic acid, 1.97 g sodium citrate and 0.695 g citric acid were dissolved in purified water to 100 g as the second blank experiment. The pH value of the mentioned second blank experiment is 5.00. In Entry 6, 2 g 3-O-ethyl ascorbic acid, 1.52 g sodium citrate and 0.926 g citric acid were dissolved in purified water to 100 g. The pH value of the solution is 4.51. In Entry 7, 2 g 3-O-ethyl ascorbic acid, 1.91 g sodium citrate and 0.64 g citric acid were dissolved in purified water to 100 g. The pH value of the solution is 5.05. In this example, different pH values (4.51 and 5.05) from the same buffer system were employed. And, the transmittance is detected at 440 nm.

As shown in Entry 6 and Entry 7 in Table 2, it can be found that lower pH value is helpful to stabilize the color of 3-O-ethyl ascorbic acid solution.

### Example 3

In this example, we try to compare the stability of ascorbic acid and ascorbic acid derivative solution with buffer. In this example, the following solutions were placed at 45° C. for 90 days, and the transmittance of the solutions on Day 0 and Day 90 were respectively detected. Table 3 presents the result of this example. In Entry 8, 2 g 3-O-ethyl ascorbic acid, 1.52 g sodium citrate and 0.926 g citric acid were dissolved in purified water to 100 g. In Entry 9, 2 g L-ascorbic acid, 2.292 g sodium citrate, 0.367 g citric acid were dissolved in purified water to 100 g. The pH value of the solutions in these examples were 4.50. In this example, the transmittance is detected at 440 nm.

As shown in Table 3, according to the color change of the samples, it is obviously that 3-O-ethyl ascorbic acid is more stable than L-ascorbic acid.

### Example 4

In this example, we try to use phosphonic acid derivative to assist stabilizing ascorbic acid derivative solution. In this example, the following solutions were placed at 45° C. for 90 days, and the transmittance of the solutions on Day 0 and Day 90 were respectively detected at 440 nm. Table 4 presents the result of this example. In Entry 10, 0 g 3-O-ethyl ascorbic acid, 1.64 g sodium citrate, 0.88 g citric acid and 0.1 g N,N,N,N-tetrakismethylene phosphonate hydrate (EDTMP) were dissolved in purified water to 100 g as blank experiment. In Entry 11, 2 g 3-O-ethyl ascorbic acid, 1.594 g sodium citrate, 0.878 g citric acid and 0.1 g N,N,N,N-tetrakismethylene phosphonate hydrate (EDTMP) were dissolved in purified water to 100 g. In Entry 12, 2 g 3-O-ethyl ascorbic acid, 1.52 g sodium citrate, 0.926 g citric acid and 0.0 g N,N,N,N-tetrakismethylene phosphonate hydrate (EDTMP) were dissolved in purified water to 100 g.

As shown in Entry 11 and Entry 12 in Table 4, according to the color change of the samples, it can be found that EDTMP is helpful to stabilize 3-O-ethyl ascorbic acid solution.

### Example 5

In this example, we try to use different concentration of alcohols to stabilize ascorbic acid derivative solution. In this example, the following solutions were placed at 45° C. for 90 days, and the transmittance of each solution on Day 0 and Day 90 were respectively detected at 440 nm. Table 6 presents the result of this example. In Entry 13, 0.00 g 3-O-ethyl ascorbic acid, 1.558 g sodium citrate; and 0.993 g citric acid were dissolved in purified water to 100 g as blank experiment. In Entry 14, 2.00 g 3-O-ethyl ascorbic acid, 1.49 g sodium citrate and 0.74 g citric acid were dissolved in purified water to 100 g. In Entry 15, 2.00 g 3-O-ethyl ascorbic acid, 1.24 g sodium citrate, 0.93 g citric acid and 10 g ethoxydiglycol were dissolved in purified water to 100 g to form a mixed well solution. In Entry 16, 2.00 g 3-O-ethyl ascorbic acid, 1.45 g sodium citrate, 0.92 g citric acid and 3.00 g butylene glycol were dissolved in purified water to 100 g to form a mixed well solution. The pH values of the solution in this example were controlled at 4.50.

### Example 6

In this example, we try to use phosphonic acid derivative and low concentration alcohols to stabilize ascorbic acid derivatives solution. In this example, the following solutions were placed at 45° C. for 90 days, and the transmittance of the solutions on Day 0 and Day 90 were respectively detected at 440 nm. Table 6A presents the result of this example. In Entry 17, 0.00 g 3-O-ethyl ascorbic acid, 1.558 g sodium citrate and 0.993 g citric acid were dissolved in purified water to 100 g as blank experiment. In Entry 18, 2.00 g 3-O-ethyl ascorbic acid, 1.52 g sodium citrate and 0.926 g citric acid were dissolved in purified water to 100 g. In Entry 19, 2.00 g 3-O-ethyl ascorbic acid, 1.594 g sodium citrate, 0.878 g citric acid and 0.1 g N,N,N,N-tetrakismethylene phosphonate hydrate (EDTMP) were dissolved in purified water to 100 g. In Entry 20, 2.00 g 3-O-ethyl ascorbic acid, 1.322 g sodium citrate, 0.86 g citric acid, 0.1 g N,N,N,N-tetrakismethylene phosphonate hydrate (EDTMP) and 10.0 g ethoxydiglycol were dissolved in purified water to 100 g. In Entry 21, 2.00 g 3-O-ethyl ascorbic acid, 1.468 g sodium citrate, 0.86 g citric acid, 0.1 g N,N,N,N-tetrakismethylene phosphonate hydrate (EDTMP) and 5.0 g ethoxydiglycol were dissolved in purified water to 100 g. In Entry 22, 2.00 g 3-O-ethyl ascorbic acid, 1.506 g sodium citrate, 0.872 g citric acid, 0.1 g N,N,N,N-tetrakismethylene phosphonate hydrate (EDTMP) and 3.0 g butylene glycol were dissolved in purified water to 100 g. The pH values of the solution in this example were controlled at 4.50.

From the above Table 6A, we can find that EDTMP and alcohols are helpful for stabilizing 3-O-ethyl ascorbic acid base on the Transmittance change, pH value and HPLC assay of the entries.

In order to compare with ascorbic acid, we also process the same test on L-ascorbic acid. The result is shown in the following Table 6B. In Entry 23, 2.00 g L-ascorbic acid, 2.292 g sodium citrate and 0.367 g citric acid were dissolved in purified water to 100 g. In Entry 24, 2.00 g L-ascorbic acid, 2.349 g sodium citrate, 0.328 g citric acid and 0.1 g N,N,N,N-tetrakismethylene phosphonate hydrate (EDTMP) were dissolved in purified water to 100 g. In Entry 25, 2.00 g L-ascorbic acid, 2.016 g sodium citrate, 0.37 g citric acid, 0.1 g N,N,N,N-tetrakismethylene phosphonate hydrate (EDTMP) and 10.0 g ethanol were dissolved in purified water to 100 g. The pH values of the solution in this example were controlled at 4.50.

From the above Table 6B, as shown in the delta Transmittance data and degradation data, we can find that EDTMP and alcohol are insufficient to stabilize L-ascorbic acid.

### Example 7

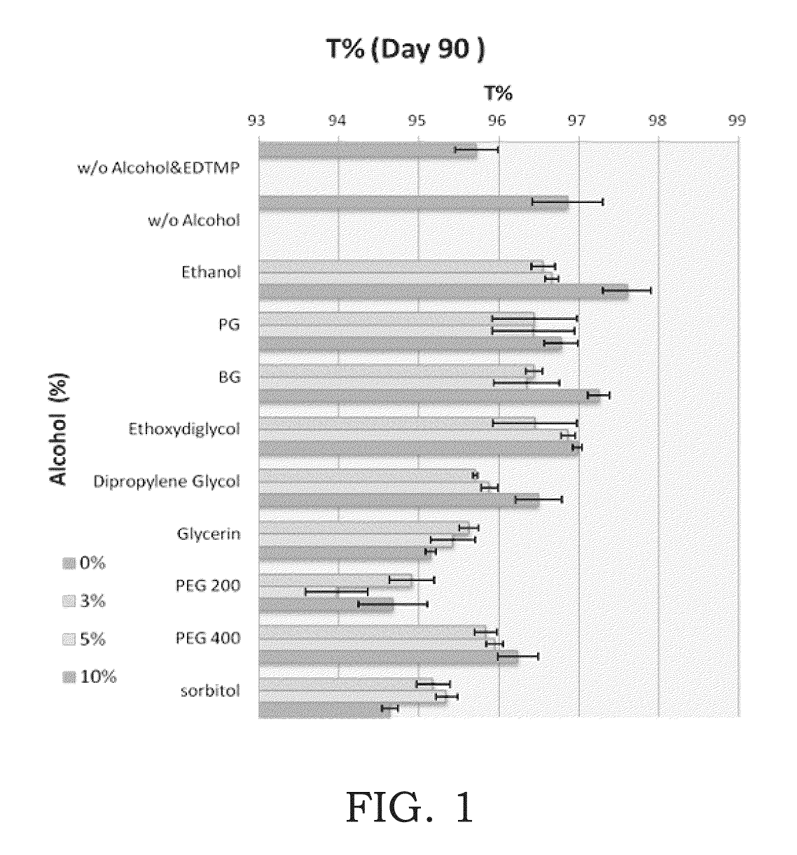

In this example, we try to use different alcohols and different amount of alcohols for stabilizing ascorbic acid derivatives solution. In this example, the following solutions were placed at 45° C. for 90 days, and the transmittance of the solutions on Day 0 and Day 90 were respectively detected at 440 nm. The pH values of the solution in this example were controlled at 4.50. Table 7 and FIG. 1 present the result of this example. In Entry 26, 2.00 g 3-O-ethyl ascorbic acid, 1.52 g sodium citrate and 0.926 g citric acid were dissolved in purified water to 100 g. In Entry 27, 2.00 g 3-O-ethyl ascorbic acid, 1.594 g sodium citrate, 0.878 g citric acid, and 0.1 g EDTMP were dissolved in purified water to 100 g. In Entry 28, 2.00 g 3-O-ethyl ascorbic acid, 1.49 g sodium citrate, 0.888 g citric acid, 0.1 g EDTMP and 3.0 g ethanol were dissolved in purified water to 100 g. In Entry 29, 2.00 g 3-O-ethyl ascorbic acid, 1.44 g sodium citrate, 0.878 g citric acid, 0.1 g EDTMP and 5.0 g ethanol were dissolved in purified water to 100 g. In Entry 30, 2.00 g 3-O-ethyl ascorbic acid, 1.294 g sodium citrate, 0.882 g citric acid, 0.1 g EDTMP and 10.0 g ethanol were dissolved in purified water to 100 g. In Entry 31, 2.00 g 3-O-ethyl ascorbic acid, 1.506 g sodium citrate, 0.872 g citric acid, 0.1 g EDTMP and 3.0 g ethoxydiglycol were dissolved in purified water to 100 g. In Entry 32, 2.00 g 3-O-ethyl ascorbic acid, 1.468 g sodium citrate, 0.86 g citric acid, 0.1 g EDTMP and 5.0 g ethoxydiglycol were dissolved in purified water to 100 g. In Entry 33, 2.00 g 3-O-ethyl ascorbic acid, 1.322 g sodium citrate, 0.86 g citric acid, 0.1 g EDTMP and 10.0 g ethoxydiglycol were dissolved in purified water to 100 g. In Entry 34, 2.00 g 3-O-ethyl ascorbic acid, 1.512 g sodium citrate, 0.866 g citric acid, 0.1 g EDTMP and 3.0 g dipropylene glycol were dissolved in purified water to 100 g. In Entry 35, 2.00 g 3-O-ethyl ascorbic acid, 1.468 g sodium citrate, 0.858 g citric acid, 0.1 g EDTMP and 5.0 g dipropylene glycol were dissolved in purified water to 100 g. In Entry 36, 2.00 g 3-O-ethyl ascorbic acid, 1.336 g sodium citrate, 0.85 g citric acid, 0.1 g EDTMP and 10.0 g dipropylene glycol were dissolved in purified water to 100 g. In Entry 37, 2.00 g 3-O-ethyl ascorbic acid, 1.506 g sodium citrate, 0.872 g citric acid, 0.1 g EDTMP and 3.0 g butylenelene glycol were dissolved in purified water to 100 g. In Entry 38, 2.00 g 3-O-ethyl ascorbic acid, 1.46 g sodium citrate, 0.864 g citric acid, 0.1 g EDTMP and 5.0 g butylenelene glycol were dissolved in purified water to 100 g. In Entry 39, 2.00 g 3-O-ethyl ascorbic acid, 1.328 g sodium citrate, 0.856 g citric acid, 0.1 g EDTMP and 10.0 g butylenelene glycol were dissolved in purified water to 100 g. In Entry 40, 2.00 g 3-O-ethyl ascorbic acid, 1.506 g sodium citrate, 0.872 g citric acid, 0.1 g EDTMP and 3.0 g propylene glycol were dissolved in purified water to 100 g. In Entry 41, 2.00 g 3-O-ethyl ascorbic acid, 1.474 g sodium citrate, 0.854 g citric acid, 0.1 g EDTMP and 5.0 g propylene glycol were dissolved in purified water to 100 g. In Entry 42, 2.00 g 3-O-ethyl ascorbic acid, 1.35 g sodium citrate, 0.84 g citric acid, 0.1 g EDTMP and 10.0 g propylene glycol were dissolved in purified water to 100 g. In Entry 43, 2.00 g 3-O-ethyl ascorbic acid, 1.506 g sodium citrate, 0.872 g citric acid, 0.1 g EDTMP and 3.0 g glycerin were dissolved in purified water to 100 g. In Entry 44, 2.00 g 3-O-ethyl ascorbic acid, 1.474 g sodium citrate, 0.854 g citric acid, 0.1 g EDTMP and 5.0 g glycerin were dissolved in purified water to 100 g. In Entry 45, 2.00 g 3-O-ethyl ascorbic acid, 1.382 g sodium citrate, 0.818 g citric acid, 0.1 g EDTMP and 10.0 g glycerin were dissolved in purified water to 100 g. In the above experiments, the total sample contains the appropriate amount of the preservative.

As shown in Table 7, 0.1% EDTMP and alcohols are helpful to stabilize the pH of 3-O-ethyl ascorbic acid solution. As shown in Entry 28 in Table 7, adding 0.1% EDTMP and 3% ethanol are helpful to stabilize pH value and assay of 3-O-ethyl ascorbic acid solution. As shown in Entry 29 in Table 7, adding 0.1% EDTMP and 5% ethanol are helpful to stabilize pH value and assay of 3-O-ethyl ascorbic acid solution. As shown in Entry 30 in Table 7, adding 0.1% EDTMP and 10% ethanol are helpful to stabilize pH value, assay and color of 3-O-ethyl ascorbic acid solution. As shown in Entry 33 in Table 7, adding 0.1% EDTMP and 10% ethoxydiglycol are helpful to stabilize pH value, color and to slow-down the degradation of 3-O-ethyl ascorbic acid solution. As shown in Entry 39 in Table 7, adding 0.1% EDTMP and 10% butylenelene glycol are helpful to stabilize pH value, assay and color of 3-O-ethyl ascorbic acid solution.

### Example 8

In this example, we try to use two alcohols in different ratios to stabilize ascorbic acid derivative solution. In this example, the following solutions were placed at 45° C. for 90 days, and the transmittance of the solutions on Day 0 and Day 90 were respectively detected at 440 nm. The pH values of the solution in this example were controlled at 4.50. In this example, in order to check the stability of ascorbic acid derivative, we used transmittance of ascorbic acid derivative solution to follow the yellowing. Furthermore, we also used HPLC to check the activity of ascorbic acid derivative from the change of the area integral of the 3-O-ethyl ascorbic acid peak in HPLC assay. Table 8 presents the result of this example. In Entry 46, 2.00 g 3-O-ethyl ascorbic acid, 1.56 g sodium citrate, 0.90 g citric acid and 0.1 g EDTMP were dissolved in purified water to 100 g. In Entry 47, 2.00 g 3-O-ethyl ascorbic acid, 1.44 g sodium citrate, 0.878 g citric acid, 0.1 g EDTMP and 5.0 g ethanol were dissolved in purified water to 100 g. In Entry 48, 2.00 g 3-O-ethyl ascorbic acid, 1.294 g sodium citrate, 0.882 g citric acid, 0.1 g EDTMP and 10.0 g ethanol were dissolved in purified water to 100 g. In Entry 49, 2.00 g 3-O-ethyl ascorbic acid, 1.46 g sodium citrate, 0.864 g citric acid, 0.1 g EDTMP and 5.0 g butylene glycol were dissolved in purified water to 100 g. In Entry 50, 2.00 g 3-O-ethyl ascorbic acid, 1.328 g sodium citrate, 0.856 g citric acid, 0.1 g EDTMP and 10.0 g butylene glycol were dissolved in purified water to 100 g. In Entry 51, 2.00 g 3-O-ethyl ascorbic acid, 1.37 g sodium citrate, 0.83 g citric acid, 0.1 g EDTMP, 5.0 g ethanol and 5.0 g butylene glycol were dissolved in purified water to 100 g. The pH values of the solution in this example were controlled at 4.50.

From the above Table 8, we can find that adding 0.1% EDTMP, 5% ethanol and 5% 1,3-butylene Glycol are helpful for stabilizing 3-O-ethyl ascorbic acid, based on the measured Transmittance data and HPLC assay. And, it also can be found that phosphonic acid derivative and at least one alcohol can be synergistic on stabilizing 3-O-ethyl ascorbic acid.

### Example 9

### Application of the Composition of Stabilizing 3-O-Ethyl Ascorbic Acid in Toner: [Water-Loving]

The following is the major components of three entries with the composition of stabilizing 3-O-ethyl ascorbic acid according to this specification.

In this example, the manufacturing of the above-mentioned toners is as the following. The part A are mixed homogeneously.

### Example 10

### Application of the Composition of Stabilizing 3-O-Ethyl Ascorbic Acid in Serum: [Water-Loving]

The following is the major components of three entries with the composition of stabilizing 3-O-ethyl ascorbic acid according to this specification.

In this example, the manufacturing of the above-mentioned serums is as the following. Part A was pre-mixed uniformly. Part B was pre-mixed uniformly. Part B and Part C ingredients were added in sequence into Part A, and then the mixture was well mixed. Part D was pre-mixed uniformly. Part D was added into Part A/B/C, and then the mixture was well mixed.

### Example 11

### Application of the Composition of Stabilizing 3-O-Ethyl Ascorbic Acid in Cream: [Oil-in-Water; O/W]

The following is the major components of the entry with the composition of stabilizing 3-O-ethyl ascorbic acid according to this specification.

In this example, the manufacturing of the above-mentioned cream is as the following. Part A and part G are pre-mixed separately. Part B was heated until it has fully melted, and then Part C was added into the melted Part B while stirring. Part D was added into part B/C, and the mixture is well-mixed. Part E was added into Part B/C/D while stirring. Part A and the mixture of part B/C/D/E are respectively heated up to 80° C. Then, the mixture of part B/C/D/E is added into part A and well mixed. The mentioned mixture of part A/B/C/D/E was stirred for 5 minutes, and then the mixture is removed from the heat source. When cooling the mixture of part A/B/C/D/E down to 40° C., part F and part G were added into the mentioned mixture sequentially, and mixed well.

### Example 12

### Application of the Composition of Stabilizing 3-O-Ethyl Ascorbic Acid in Cream: [Oil-in-Water; O/W]

The following is the major components of the entry with the composition of stabilizing 3-O-ethyl ascorbic acid according to this specification.

The manufacturing of the above-mentioned cream is as the following. Part A and part G are pre-mixed separately. Part B was heated until it has fully melted, and then Part C was added into Part B while stirring. Part D was added into part B/C, and the mixture is well-mixed. Part E was added into Part B/C/D while stirring. Part A and the mixture of part B/C/D/E are respectively heated up to 80° C. Then, the mixture of part B/C/D/E was added into part A and mixed well. The mentioned mixture of part A/B/C/D/E are stirred for 5 minutes, and then the mixture is removed from the heat source. When cooling the mixture of part A/B/C/D/E down to 40° C., part F and part G are added into the mentioned mixture sequentially, and mixed well.

### Example 13

### Application of the Composition of 10% 3-O-Ethyl Ascorbic Acid Cream (O/W) with 0.1% EDTMP

The following is the major components of the entry with the composition of stabilizing 3-O-ethyl ascorbic acid according to this specification.

The manufacturing of the above-mentioned cream is as the following. Heat Part B until it has fully melted, and then add Part C into Part B while stirring. Part D is added into part B/C, and the mixture is well-mixed. Add Part E into Part B/C/D while stirring. Part A and the mixture of part B/C/D/E are respectively heated up to 80° C. Then, the mixture of part B/C/D/E is added into part A and mixed well. The mentioned mixture of part A/B/C/D/E are stirred for 5 minutes, and then the mixture is removed from the heat source. When cooling the mixture of part A/B/C/D/E down to 40° C., part F is added into the mentioned mixture sequentially, and mixed well.

In summary, we have reported a method for stabilizing ascorbic acid derivatives and the application thereof. The method comprises mixing ascorbic acid derivative with a composition, wherein the composition comprises buffer, phosphonic acid derivative and at least one alcohol. The composition can be selected from an oil-in-water composition, and a water-loving composition. According to this invention, we find out that the stability of ascorbic acid derivative can be improved by adding buffer, phosphonic acid derivative, or alcohol separately. We also find out that when forming a composition comprising buffer, phosphonic acid derivative, and alcohol, the stabilizing effect can be synergistic. Preferably, all the components in the mentioned method for stabilizing ascorbic acid derivatives are not expensive, so that it will not raise the cost too much while employing the mentioned method to replace L-ascorbic acid in cosmetics and dermatologic fields. More preferably, the method is mild, so that it can be applied in cosmetics and dermatologic fields without allergic sensitizations and irritant reactions to human skin.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for stabilizing ascorbic acid derivatives, wherein a general formula of the ascorbic acid derivative is as the following, 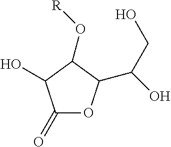
comprising:
mixing the ascorbic acid derivatives with a composition, wherein said composition comprises:
a buffer, wherein the buffer is employed to adjust pH value of the composition between 3.5 and 5.5; and
a stabilizing agent consisted of phosphonic acid derivative; and
alcohol, wherein said alcohol is selected from one or the combination of the group consisting of the following: ethanol, propylene glycol, 1,3-propanediol, dipropylene glycol, butylene glycol, ethoxydiglycol, and polyethylene glycol (PEG), wherein said stabilizing agent does not include dimethiconecopolyol and alkyldimethiconecopolyol;
wherein R is selected from one of the group consisting of the following: C1-C20 alkyl group.

2. The method for stabilizing ascorbic acid derivatives according to claim 1, wherein said buffer is selected from one of the group consisting of the following: citric acid/sodium citrate, citric acid/sodium phosphate, and acetic acid/sodium acetate.

3. The method for stabilizing ascorbic acid derivatives according to claim 1, wherein said phosphonic acid derivative is selected from one of the group consisting of the following: N,N,N′,N′-ethylenediaminetetrakis(methylenephosphonic acid) hydrate (EDTMP), hexaMethylenediaminetetra(methylenephosphonic Acid) (HMDTMPA), Diethylene Triamine Penta(Methylene Phosphonic Acid) (DTPMPA) and the salts thereof.

4. The method for stabilizing ascorbic acid derivatives according to claim 1, wherein said composition is an oil-in-water composition.

5. The method for stabilizing ascorbic acid derivatives according to claim 1, wherein said composition is a water-loving composition.

6. The method for stabilizing ascorbic acid derivatives according to claim 1, wherein the quantity of the ascorbic acid derivative is from 0.01 to 10% of the total weight of the composition, wherein the quantity of the buffer is not more than 6.45% of the total weight of the composition.

7. The method for stabilizing ascorbic acid derivatives according to claim 1, wherein the quantity of the ascorbic acid derivative is from 0.1 to 4.0% of the total weight of the composition, wherein the quantity of the buffer is not more than 6.45% of the total weight of the composition.

8. The method for stabilizing ascorbic acid derivatives according to claim 1, wherein the quantity of the phosphonic acid derivative is from 0.01 to 1.0% of the total weight of the composition.

9. The method for stabilizing ascorbic acid derivatives according to claim 1, wherein the quantity of the phosphonic acid derivative is from 0.1 to 0.5% of the total weight of the composition.

10. The method for stabilizing ascorbic acid derivatives according to claim 1, wherein the quantity of the alcohol is not more than 20% of the total weight of the composition.

11. The method for stabilizing ascorbic acid derivatives according to claim 1, wherein the quantity of the alcohol is not more than 10% of the total weight of the composition.

12. The method for stabilizing ascorbic acid derivatives according to claim 1, wherein the pH value of the composition is between 3.8 and 4.5.

13. The method for stabilizing ascorbic acid derivatives according to claim 1, wherein said ascorbic acid derivative is 3-O-ethyl ascorbic acid with the structure as following,

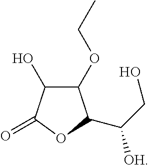

14. An oil-in-water composition for stabilizing ascorbic acid derivatives, wherein a general formula of the ascorbic acid derivative is as the following, 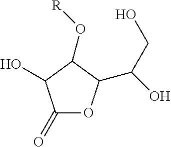
comprising:
a buffer, wherein the buffer is employed to adjust pH value of the composition between 3.5 and 5.5;
a stabilizing agent consisted of phosphonic acid derivative; and
alcohol, wherein said alcohol is selected from one or the combination of the group consisting of the following: ethanol, propylene glycol, 1,3-propanediol, dipropylene glycol, butylene glycol, ethoxydiglycol, and polyethylene glycol (PEG);
wherein R is selected from one of the group consisting of the following: C1-C20 alkyl group.

15. The composition for stabilizing ascorbic acid derivatives according to claim 14, wherein said buffer is selected from one of the group consisting of the following: citric acid/sodium citrate, citric acid/sodium phosphate and acetic acid/sodium acetate.

16. The composition for stabilizing ascorbic acid derivatives according to claim 14, wherein the quantity of the ascorbic acid derivative is from 0.01 to 10% of the total weight of the composition, wherein the quantity of the buffer is not more than 6.45% of the total weight of the composition.

17. The composition for stabilizing ascorbic acid derivatives according to claim 14, wherein the quantity of the phosphonic acid derivative is from 0.01 to 1.0% of the total weight of the composition.

18. The composition for stabilizing ascorbic acid derivatives according to claim 14, wherein the quantity of the phosphonic acid derivative is from 0.1 to 0.5% of the total weight of the composition.

19. The composition for stabilizing ascorbic acid derivatives according to claim 14, wherein the quantity of the alcohol is not more than 20% of the total weight of the composition.

20. The composition for stabilizing ascorbic acid derivatives according to claim 14, wherein the quantity of the alcohol is not more than 10% of the total weight of the composition.

21. The composition for stabilizing ascorbic acid derivatives according to claim 14, wherein the pH value of the composition is between 3.8 and 4.5.

22. The composition for stabilizing ascorbic acid derivatives according to claim 14, further comprising water.

23. The composition for stabilizing ascorbic acid derivatives according to claim 14, wherein said ascorbic acid derivative is 3-O-ethyl ascorbic acid with the structure as following, [chemical structure]
